(19) United States
(12) Patent Application Publication
(10) Pub. No.: US 2026/0008981 A1
(43) Pub. Date: Jan. 8, 2026

(54) METHODS FOR PRODUCING BIOGAS FROM A FIBROUS SUBSTRATE

(71) Applicant: NOVOBIOM, Liege (BE)

(72) Inventors: Maxime Dessily, Liege (BE); Caroline Zaoui, Liege (BE)

(21) Appl. No.: 19/335,507

(22) Filed: Sep. 22, 2025

Related U.S. Application Data
(63) Continuation-in-part of application No. PCT/EP2024/057466, filed on Mar. 20, 2024.
(63) Continuation-in-part of application No. PCT/EP2024/057468, filed on Mar. 20, 2024.

(30) Foreign Application Priority Data
Mar. 20, 2023 (BE) 2023/5203
Mar. 20, 2023 (BE) 2023/5206

(51) Int. Cl. C12M 1/107; C12P 5/02; D06M 16/00

(52) U.S. Cl. CPC C12M 21/04; C12P 5/023; D06M 16/003

(57) ABSTRACT

Method for producing biogas from a fibrous substrate comprising biological pretreatment by solid-state fermentation using one or more filamentous fungal strains, more particularly using one or more saprophytic filamentous strains, a woven, non-woven, or agglomerated, and shredded fibrous substrate, biogas production from the pretreated fibrous substrate with formation of a digestate, and digestate collection.

This invention relates to a method for producing biogas from a fibrous substrate, comprising:
Biologically pretreating the fibrous substrate, forming a biologically pretreated fibrous substrate
Transferring the biologically pretreated fibrous substrate to a biogas production facility
Producing biogas from the pretreated fibrous substrate with the formation of a digestate,
Collecting the digestate

Such biogas production methods are well known in the state of the art, as is the case for the production of biogas from plant waste or wood waste.

In this case, biological pretreatment has been implemented to replace mechanical or chemical pretreatments and involves pretreating waste using enzymes to facilitate attack by methanogenic microorganisms in the biogas production facility.

Indeed, mechanical/physical or chemical pretreatments are often necessary to achieve biogas production yields that make the facility profitable. Unfortunately, these pretreatments still require energy resources (crushing, heating, pressurising) or products such as bases, sometimes strong bases, or acids to carry out preliminary attacks on waste. In the case of chemical treatments, even though significant efforts have been made to recycle these basic and acidic liquid flows, these treatments still consume water and produce liquid phases that also require treatment. Furthermore, pretreated waste often requires further treatment before it can enter the biogas production facility (neutralisation, heat treatment, etc.). It is for these reasons, among others, that some authors have turned to biological pretreatments using enzymes, which have proven fairly effective in increasing biogas production from waste, but which generate significant additional production costs. Enzymes are relatively expensive substances, and to function optimally, they also require fairly strict temperature and pH conditions, making their industrial use risky and currently uncommon for most substrates suitable for biogas production. Authors generally report that they are used for complex substrates, where carbonaceous material is not easily accessible to bacteria in the biogas production facility.

Substrates where carbonaceous material is difficult to access include wood waste, but also textile waste, which has become a major challenge in waste management.

In 2021, more than 149 million tonnes of textiles were produced worldwide. In Europe, textile consumption has increased by more than 40% in the past two decades, and it is estimated that each inhabitant throws away an average of 11 kg of textiles per year. Only 38% of textile waste is collected and sorted for potential reuse, and it is estimated that only 1% is recycled and 87% is incinerated or buried in landfills.

One of the major problems with textile waste lies in how varied it is. Indeed, there are not only different types of waste, but also many types of textiles, and within these, there are many types of fibre that are blended to form the textiles.

While some solutions currently exist for textile production waste (cutting waste, defective materials, production leftovers, etc.), the nature of which is well known and controlled, some types of textile waste are extremely problematic in terms of recycling.

At the moment, for textiles from clothing, existing solutions involve mechanical sorting to recover garments that can be sold second-hand. For other textiles, more advanced sorting to separate fabrics based on their composition is carried out manually or by fibre optics. Textile waste from furniture is currently not recycled and is used for its heat-generating potential.

Furthermore, blends of synthetic and natural fibres found in ticking and other net curtains are a source of plastic fibres which cannot be exploited using existing techniques, despite the existence of physical-chemical treatments to weaken the natural fibres so they can be spun.

Moreover, while in some cases, mattress ticking is separated from the rest of the mattress, this is not always the case, and this depends on the technology available to the waste recovery centres responsible for recycling. Also, some centres crush mattresses with their ticking, remove the metal parts, and thus generate a shredded material comprising a mixture of shredded ticking and synthetic or natural foam.

The invention aims to overcome the disadvantages of the state of the art by providing a method for producing biogas that provides a way of recovering complicated waste such as mattress waste, upholstery waste, end-of-life textile waste, textile waste from furnishing fabrics, etc.

To solve this problem, the invention provides a method for producing biogas as indicated at the start, characterised in that the biological pretreatment of the fibrous substrate is solid-state fermentation using one or more filamentous fungal strains, more particularly one or more saprophytic filamentous fungal strains, even more particularly chosen from the Basidiomycetes and Ascomycetes divisions, of a woven, non-woven or agglomerated fibrous substrate, and shredded into pieces, where said biologically pretreated fibrous substrate is a fibrous substrate shredded into pieces colonised by said at least one fungal strain.

As can be seen, the method according to this invention provides a biological pretreatment of the fibrous substrate which has several advantages. First, pretreatment by solid-state fermentation using one or more fungal strains removes the need for large quantities of water, large volumes of chemicals, or expensive reagents, and provides a structural solution without requiring a significant investment in infrastructure. Fungal strains are known for their ability to digest compounds that are particularly difficult to access, or even particularly polluting or toxic, and their action opens up fibre structures to facilitate the action of methanogenic microorganisms. In addition, the fungal strain(s) feed on carbonaceous materials so they can grow and thus reduce the proportion of complex and inaccessible fibres in the fibrous substrate by producing a biomass that is easier for methanogenic microorganisms to digest, namely mycelia and other fungal compounds. Therefore, the proportion of complex substrate for digestion on the accessible substrate is more favourable after pretreatment with one or more fungal strains.

Advantageously, in the method according to this invention, said one or more fungal strains comprises at least one fungal strain with high colonising power chosen from the group consisting of the genera Agrocybe, Ganoderma, Trametes, Pycnoporus, Pleurotus, Fomes, Fomitopsis, Irpex, Laetiporus, Inonotus, Lentinula, Fusarium, Aspergillus, Trichoderma, Penicillium, Cladosporium, Chaetomium, Acremonium. In particular, the following species are preferred: Agrocybe sp., Ganoderma sp., G. applanatum, G. boninense, G. lucidum, G. resinaceum, G. sessile, Trametes sp.; Trametes hirsuta, T. pubescens, T. suaveolens, T. versicolor, Pycnoporus sanguineus, Pleurotus sp., P. albidus, P. citrinopileatus, P. djamor, P. eryngii, P. ostreatus, P. ostraceus florida, P. ostraceus sajorcaju caju, P. salmoneo-stramineus, Fomes fomentarius, Fomitopsis pinicola, Irpex lacteus, Laetiporus sulphureus, Inonotus obliquus, Lentinula edodes; Lentinus lepideus, L. giganteus, L. squarrosulus and L. tigrinus, Fusarium sp., Fusarium culmorum, Fusarium solani, Aspergillus sp., Aspergillus oryzae, Aspergillus niger, Aspergillus flavus, Aspergillus fumigatus, Aspergillus terreus, Trichoderma sp., Trichoderma reesei, Trichoderma viride, Trichoderma longibrachiatum, Cladosporum sp., Chaetomium sp., Chaetomium globosum. This provides fungal strains that spread widely and provide a large amount of biomass that can be used by methanogenic microorganisms, either for methane production or as a culture substrate. Moreover, during their colonisation, fungal strains use the carbonaceous material found in the fibrous substrate and begin digesting it. In this way, the at least one fungal strain allows for the conversion of poorly accessible carbonaceous material into carbonaceous material that is more easily accessible to methanogenic microorganisms.

Advantageously according to this invention, said one or more fungal strains comprises at least one enzyme-producing fungal strain chosen from the group consisting of strains belonging to the genera Agrocybe, Ganoderma, Trametes, Pycnoporus, Pleurotus, Fomes, Fomitopsis, Irpex, Laetiporus, Inonotus, Lentinula, Fusarium, Aspergillus, Trichoderma, Penicillium, Cladosporium, Chaetomium, Acremonium, more particularly chosen from the strains Agrocybe sp., Ganoderma sp., G. applanatum, G. boninense, G. lucidum, G. resinaceum, G. sessile, Trametes sp.; Trametes hirsuta, T. pubescens, T. suaveolens, T. versicolor, Pycnoporus sanguineus, Pleurotus sp., P. albidus, P. citrinopileatus, P. djamor, P. eryngii, P. ostreatus, P. ostraceus florida, P. ostraceus sajorcaju caju, P. salmoneo-stramineus, Fomes fomentarius, Fomitopsis pinicola, Irpex lacteus, Laetiporus sulphureus, Inonotus obliquus, Lentinula edodes; Lentinus lepideus, L. giganteus, L. squarrosulus and L. tigrinus, Fusarium sp., Fusarium culmorum, Fusarium solani, Aspergillus sp., Aspergillus oryzae, Aspergillus niger, Aspergillus flavus, Aspergillus fumigatus, Aspergillus terreus, Trichoderma sp., Trichoderma reesei, Trichoderma viride, Trichoderma longibrachiatum, Cladosporum sp., Chaetomium sp., Chaetomium globosum. The enzymes produced in this case may have various uses; either they contribute to the degradation of the fibrous substrate, or the enzymes are co-products of fungal attack on the fibrous substrate which can be collected and then purified for subsequent sale.

In some cases, according to this invention, it is envisaged that said one or more fungal strains are a mixture of one or more strains with high colonising power and one or more enzyme-producing strains.

In the context of this invention, “biogas” preferably means a biogenic gas, preferably a gas comprising methane, optionally other gases that can be used as fuel, and carbon dioxide, the latter advantageously being post-treated (capturing, supercritical extraction, reaction to form other molecules).

In a preferred embodiment according to this invention, wherein the agglomerated, woven, or non-woven shredded fibrous substrate comprises fibres chosen from natural plant or animal textile fibres, semi-synthetic textile fibres or polymer textile fibres, and lignocellulosic fibres.

The term natural plant textile fibres means, within the meaning of this invention, natural textile fibres of abaca, bagasse, bamboo, coconut, cotton, linen, hemp, jute, raffia, ramie, rattan, wood, Furcraea andina, Ceiba pentandra, Agave sisalana, Kenaf, Piña, and the like.

The term natural animal textile fibres means natural textile fibres of alpaca, angora, byssus, camel hair, cashmere, catgut, guanaco, hair or fur, llama, mohair, pashmina, qiviuk, silk, possibly spider silk, sinew, wool, vicuna, yak and the like,

For the purposes of this invention, the term semi-synthetic fibres means fibres made of cellulose acetate, cellulose diacetate, cellulose triacetate, lyocell, modal, and the like.

The term polymer textile fibres means acrylic fibres, aramid fibres (Twaron, Kevlar, Nomex, Technora), microfibres, polyamide fibres, polyester fibres, polyolefin fibres, high molecular weight polyethylene fibres, elastane fibres, vectran fibres, vinalon fibres, zylon fibres, and the like.

This classification was published by Weidmann in 2010.

For the purposes of this invention, the term lignocellulosic fibres means fibres composed of lignin, hemicellulose, and cellulose in varying proportions, derived from forestry, agricultural operations, and waste (furniture wood, chipboard, etc.)

The terms “shredded fibrous substrate, formed from said fibres in agglomerated, woven, or non-woven form,” or “fibrous substrate in the form of pieces or granules formed from said fibres in agglomerated, woven, or non-woven form,” mean agglomerated, woven, or non-woven fibres, typically textile or lignocellulosic fibres, which have undergone a step to reduce their size in order to form pieces. This size reduction may comprise crushing using a shearing crusher, a guillotine cutter, a shredder, a jaw crusher, or even a tearing machine. This crushing step takes place before the supply of fibrous substrate. In certain cases, prior to the moistening step, this invention also envisages performing an additional size reduction step if this proves useful, for example when the size distribution of the fibrous substrate in the form of pieces or granules formed from said fibres is too large or when the average size of the pieces is too large.

Advantageously, said fibrous substrate in agglomerated, woven, or non-woven form is a residue from the crushing of recycled textiles, more particularly recycled furnishing textiles, recycled mattresses, bathroom or bed linen, clothing textiles, textile production scraps or waste, upholstery, and mixtures thereof.

The term upholstered refers to products such as cushions, sofa seats, and soft toys, namely products typically containing a mixture of textile material and upholstery foam.

More particularly, according to this invention, said fibrous substrate in agglomerated, woven or non-woven form is a residue from the crushing of recycled textiles chosen from furnishing, mattress and upholstery textiles and has a synthetic foam content of between 10 and 80%.

In another embodiment according to this invention, said fibrous substrate in agglomerated, woven or non-woven form is a residue from the crushing of lignocellulosic elements, such as for example residue from the crushing of chipboard.

In a preferred embodiment of this invention, said fibrous substrate comprises a proportion of synthetic fibres, semi-synthetic fibres, synthetic foam, such as for example PU foam, and natural plant or animal fibres, and wherein the collected digestate is enriched with synthetic fibres and plastic materials at a rate of more than 85% by weight relative to the weight of digestate.

In another preferred embodiment according to this invention, the biological pretreatment step comprises:
conditioning said fibrous substrate to obtain a conditioned fibrous substrate containing from 60 to 80% by weight of water relative to the weight of conditioned fibrous substrate,
sanitising said conditioned fibrous substrate to form a sanitised conditioned fibrous substrate,
cooling the sanitised conditioned fibrous substrate for a period of time of between 12 and 24 hours,
inoculating said sanitised conditioned fibrous substrate with mycelium spawn from said one or more fungal strains on seeds by adding at least one mycelium spawn on seeds to said sanitised conditioned fibrous substrate at a rate of from 0.5% to 10% by weight, more particularly from 1 to 7%, even more particularly from 3 to 5% by weight of mycelium spawn relative to the weight of sanitised conditioned fibrous substrate, obtaining an inoculated sanitised conditioned fibrous substrate,
mixing said inoculated sanitised conditioned fibrous substrate to obtain a homogenised inoculated sterilised conditioned fibrous substrate,
incubating said homogenised inoculated sterilised conditioned fibrous substrate for a period of time of between 1 and 6 weeks, more particularly of between 2 and 5 weeks, in an enclosure having a relative humidity of between 65 and 85%, more particularly of between 70 and 80%,
collecting a fibrous substrate colonised by said one or more fungal strains forming said biologically pretreated fibrous substrate.

As an alternative to, or in addition to, mycelium spawn, a liquid culture of said one or more fungal strains is used for inoculating.

In this case, the concentration for inoculating will advantageously be determined by a person skilled in the art, taking into account the concentration of the starting liquid culture and the growth capacity of the strain on the fibrous substrate to be treated. Typically, between 1 and 5% (weight: weight) of the liquid medium is used for inoculating the sanitised conditioned fibrous substrate.

Advantageously, in the method according to this invention, said sanitisation of the conditioned fibrous substrate is pasteurisation so as to obtain the sanitised conditioned fibrous substrate for a period of at least 3 hours, preferably at least 4 hours, more preferably at least 5 hours at a temperature of greater than or equal to 72° C.

More particularly, in the method according to this invention, pasteurisation is carried out at increasing temperature until a peak temperature of greater than 85° C. is obtained, more particularly 88° C., more particularly 90° C., maintained for a period of time of between 5 and 50 minutes, more particularly 30 and 40 minutes.

In a variant according to this invention, said sanitisation of the conditioned fibrous substrate is a composting comprising at least one composting cycle comprising a step of increasing the temperature until a temperature of between 55 and 80° C. is obtained, more preferably until a temperature of between 58 and 65° C. is obtained, for a period of time of between 6 hours and 5 days, followed by a step of ventilating said fibrous substrate to maintain a temperature of between 46 and 49° C. for 3 to 7 days, optionally by turning the fibrous substrate over.

In a preferred embodiment according to this invention, said step of conditioning said fibrous substrate to obtain a conditioned fibrous substrate comprises moistening the fibrous substrate and/or washing said fibrous substrate optionally followed by draining or drying.

In yet another preferred embodiment according to this invention, said step of conditioning said fibrous substrate comprises an additional step of supplementing with essential elements, such as minerals (calcium, magnesium), phosphorus, carbon and nitrogen sources, typically to obtain a fibrous substrate whose carbon: nitrogen ratio is between 10 and 30, preferably between 15 and 20, for example by adding grains.

In another variant according to this invention, said sanitisation of the conditioned fibrous substrate comprises at least 2, 3, 4, 5, 6, 7, 8, or even 10 consecutive composting cycles.

In an advantageous embodiment according to this invention, the method comprises, simultaneously with pretreatment, a production of biomolecules, collected in parallel, said one or more fungal strains comprising at least one biomolecule-producing strain.

In an advantageous embodiment of this invention, said biomolecules are sugars or polysaccharides and wherein said at least one fungal strain comprises at least one sugar or polysaccharide-producing strain chosen from the group of strains of the genera Agrocybe, Ganoderma, Trametes, Pycnoporus, Pleurotus, Fomes, Fomitopsis, Irpex, Laetiporus, Inonotus, Lentinula, Fusarium, Aspergillus, Trichoderma, Penicillium, Cladosporium, Chaetomium, Acremonium.

The polysaccharides produced may be, by way of non-limiting example, α-glucans, β-glucans, lentanins, lipopolysaccharides, PSK (Polysaccharide Krestin), PSP (Polysaccharide peptide), β-d-glucans, glucuronoglucans.

In an advantageous embodiment according to this invention, said biomolecules are biomolecules of therapeutic or pharmaceutical interest or precursors thereof such as, for example, antibiotics, antimitotics, antivirals, biosorbents, biosurfactants and wherein said at least one fungal strain comprises at least one strain producing therapeutic biomolecules or suitable for pharmaceutical processes such as, for example, antibiotics, antimitotics, antivirals, biosorbents, biosurfactants chosen from the group of strains of the genera Aspergillus, Trichoderma, Penicillium, Fusarium, Pleurotus, Pycnoporus, Trametes, Ganoderma.

For example, fungi of the genus Ganoderma are composed of triterpenoids and polysaccharides. Triterpenoids have been reported to exhibit hepatoprotective effects, acting against hypertension, hypocholesterolemic and anti-histamine effects, and they also exhibit anti-tumour and antiangiogenic activity, anti-platelet aggregation effects and complement inhibitory effects. Examples of triterpenoids include ganodermic acids, lucidenic acids, ganoderic acids, ganolucidic and applanoxidic acids, lucidimols A and B, ganodermanondiol, ganoderiol F, and gano-dermanontriol, lucidones. Polysaccharides, in turn, have also been reported to exhibit anti-tumour effects through immunomodulation and anti-angiogenesis. Polysaccharides also have a protective effect against free radicals and can reduce cellular damage caused by mutagenic agents. Some polysaccharides have also been reported to have anti-diabetic effects.

Besides polysaccharides, oligosaccharides can be produced by selected microorganisms. In the context of the present invention, by “oligosaccharides”, it is preferably meant saccharides comprising from 3 to 10 sugar monomers.

Trametes versicolor extracts exhibit anti-radical, anti-oxidant, anti-bacterial and acetylcholinesterase inhibition activities.

Extracts from fungi of the genus Pleurotus exhibit therapeutic effects such as hypocholesterolemic, free radical scavenging, anti-oxidant, anti-atherogenic, anti-tumour, and immunomodulatory effects. Fungi of the genus Pleurotus, for example, have been reported to contain triterpenoids such as 2,3,6,23-tetrahydroxy-urs-12-en-28 oic acid, 2,3,23-trihydroxyurs-12-en-28 oic acid, and lupeol.

Pigments from fungi of the genus Pycnoporus exhibit anti-viral, anti-bacterial, and anti-inflammatory properties.

Fungi of the genera Aspergillus, Trichoderma, and Penicillium have been reported to contain biosurfactants.

Fungi of the genera Penicillium, Acremonium, and Aspergillus have been reported to secrete anti-biotics such as penicillin and cephalosporin.

In another advantageous embodiment according to this invention, said biomolecules are proteins and/or enzymes and wherein said at least one fungal strain comprises at least one enzyme-producing strain chosen from the group of strains belonging to the genera Agrocybe, Ganoderma, Trametes, Pycnoporus, Pleurotus, Fomes, Fomitopsis, Irpex, Laetiporus, Inonotus, Lentinula, Fusarium, Aspergillus, Trichoderma, Penicillium, Cladosporium, Chaetomium, Acremonium, more particularly chosen from the strains Agrocybe sp., Ganoderma sp., G. applanatum, G. boninense, G. lucidum, G. resinaceum, G. sessile, Trametes sp.; Trametes hirsuta, T. pubescens, T. suaveolens, T. versicolor, Pycnoporus sanguineus, Pleurotus sp., P. albidus, P. citrinopileatus, P. djamor, P. eryngii, P. ostreatus, P. ostraceus florida, P. ostraceus sajorcaju caju, P. salmoneo-stramineus, Fomes fomentarius, Fomitopsis pinicola, Irpex lacteus, Laetiporus sulphureus, Inonotus obliquus, Lentinula edodes; Lentinus lepideus, L. giganteus, L. squarrosulus and L. tigrinus, Fusarium sp., Fusarium culmorum, Fusarium solani, Aspergillus sp., Aspergillus oryzae, Aspergillus niger, Aspergillus flavus, Aspergillus fumigatus, Aspergillus terreus, Trichoderma sp., Trichoderma reesei, Trichoderma viride, Trichoderma longibrachiatum, Cladosporium sp., Chaetomium sp., Chaetomium globosum.

More particularly, the enzymes are chosen from the group formed by proteases, laccases, amylases, cellulases, chitinases, xylanases, manganese peroxidases, lipases, lignin peroxidases.

Besides enzymes, proteins with no enzyme activity can be produced by selected strains. On the basis of the disclosure of the present invention, the skilled person will readily identify which protein to ferment and how to do so.

In yet another advantageous embodiment according to this invention, said biomolecules are active biomolecules such as, for example, UV filters, pigments, anti-oxidants, anti-radical substances, and wherein said at least one fungal strain comprises at least one strain producing active biomolecules and is chosen from the group of strains belonging to the genera Pycnoporus, Pleurotus, Trametes, Fusariums, Ganoderma, such as, for example, Pycnoporus sanguineus, Pleurotus citrinopileatus, Fusarium oxysporum, Fusarium graminearum, Fusarium fujikuroi, Trametes versicolor.

Examples of pigments include, but are not limited to, cinnabarin, cinnabarinic acid, tramesanguine, PsPCP, carmine red anthraquinone, aurofusarin, and bikaverin.

Other embodiments of the method for producing biogas according to the invention are indicated in the appended claims as well as in the description below, by way of non-limiting example.

This invention relates to a method for producing biogas from a fibrous substrate wherein a fibrous substrate is biologically pretreated by solid-state fermentation using one or more fungal strains, thereby forming a fibrous substrate shredded into pieces colonised by said at least one fungal strain and which is then transferred to a biogas production facility. Once transferred to the biogas production facility, the microorganisms in the biogas production facility can then digest (anaerobic digestion) the fibrous substrate colonised by said one or more fungal strains.

The digestate thus produced after biogas production is then collected.

According to this invention, the fungal strain may be a strain with high colonising power that will rapidly grow on the fibrous substrate and use it for its growth. This makes it possible to convert carbonaceous material that is difficult for methanogenic microorganisms to access into more easily accessible carbonaceous material. The fungal strain used may also be an enzyme-producing strain. In this case, simultaneously with the growth of the fungal biomass, enzyme production occurs, which thus acts in a synergistic manner on the digestion of the fibres of the fibrous substrate. The enzymes facilitate subsequent access to methanogenic microorganisms.

In certain embodiments according to the invention, a mixture of several strains will be chosen in order to optimise the pretreatment of the fibrous substrate by the fungal strains.

The agglomerated, woven, or non-woven shredded fibrous substrate comprises fibres chosen from natural plant or animal textile fibres, semi-synthetic textile fibres or polymer textile fibres, and lignocellulosic fibres. This preferably involves a residue from the crushing of recycled textiles, more particularly recycled furnishing textiles, recycled mattresses, bathroom or bed linen, clothing textiles, textile production scraps or waste, upholstery, and mixtures thereof. This even more preferably involves a residue from the crushing of recycled textiles chosen from furnishing, mattress and upholstery textiles and has a synthetic foam content of between 10 and 80%. This residue may also contain wood chipping residues.

In the specific case of upholstery fabrics, mattresses, and upholstery, there is currently no recovery method, as this waste contains too many different compositions. Typically, the fibrous substrate includes a proportion of synthetic fibres, semi-synthetic fibres, synthetic foam, such as PU foam, and natural plant or animal fibres.

While many stakeholders view the presence of synthetic fibres or contamination by plastic foams, such as PU foam, as a disadvantage because this typically involves a substrate that cannot be digested by methanogenic microorganisms, this invention is a technological breakthrough since it chooses to treat the fibrous substrate with synthetic contaminants in order to enrich the digestate with synthetic and plastic materials so that it can be recovered in the plastics recycling sector. According to this invention, the collected digestate is enriched with synthetic fibres and plastic materials at a rate of more than 85% by weight relative to the weight of the digestate.

According to this invention, the biological pretreatment step comprises:
conditioning said fibrous substrate to obtain a conditioned fibrous substrate containing from 60 to 80% by weight of water relative to the weight of conditioned fibrous substrate,
sanitising said conditioned fibrous substrate to form a sanitised conditioned fibrous substrate,
cooling the sanitised conditioned fibrous substrate for a period of time of between 12 and 24 hours,
inoculating said sanitised conditioned fibrous substrate with mycelium spawn from said one or more fungal strains on seeds by adding at least one mycelium spawn on seeds to said sanitised conditioned fibrous substrate at a rate of from 0.5% to 10% by weight, more particularly from 1 to 7%, even more particularly from 3 to 5% by weight of mycelium spawn relative to the weight of sanitised conditioned fibrous substrate, obtaining an inoculated sanitised conditioned fibrous substrate,
mixing said inoculated sanitised conditioned fibrous substrate to obtain a homogenised inoculated sterilised conditioned fibrous substrate,
incubating said homogenised inoculated sterilised conditioned fibrous substrate for a period of time of between 1 and 6 weeks, more particularly of between 2 and 5 weeks, in an enclosure having a relative humidity of between 65 and 85%, more particularly of between 70 and 80%, and
collecting a fibrous substrate colonised by said one or more fungal strains forming said biologically pretreated fibrous substrate.

This makes it possible to use fibrous substrates that are not currently recovered due to the difficulty in accessing the carbon mass.

According to this invention, said sanitisation of the conditioned fibrous substrate is pasteurisation so as to obtain the sanitised conditioned fibrous substrate for a period of at least 3 hours, preferably at least 4 hours, more preferably at least 5 hours at a temperature greater than or equal to 72° C.

More particularly, in the method according to this invention, pasteurisation is carried out at increasing temperature until a peak temperature of greater than 85° C. is obtained, more particularly 88° C., more particularly 90° C., maintained for a period of time of between 5 and 50 minutes, more particularly 30 and 40 minutes.

In a variant according to this invention, said sanitisation of the conditioned fibrous substrate is a composting comprising at least one composting cycle comprising a step of increasing the temperature until a temperature of between 55 and 80° C. is obtained, more preferably until a temperature of between 58 and 65° C. is obtained, for a period of time of between 6 hours and 5 days, followed by a step of ventilating said fibrous substrate to maintain a temperature of between 46 and 49° C. for 3 to 7 days, optionally by turning the fibrous substrate over.

Composting indeed provides sufficient sanitisation of the fibrous substrate while removing the need for energy-intensive steps.

In a preferred embodiment according to this invention, said step of conditioning said fibrous substrate to obtain a conditioned fibrous substrate comprises moistening the fibrous substrate and/or washing said fibrous substrate optionally followed by draining or drying.

In yet another preferred embodiment according to this invention, said step of conditioning said fibrous substrate comprises an additional step of supplementing with essential elements, such as minerals (calcium, magnesium), phosphorus, carbon and nitrogen sources, typically to obtain a fibrous substrate whose carbon: nitrogen ratio is between 10 and 30, preferably between 15 and 20, for example by adding grains.

In another variant according to this invention, said sanitisation of the conditioned fibrous substrate comprises at least 2, 3, 4, 5, 6, 7, 8, or even 10 consecutive composting cycles.

The method according to this invention also aims to produce biomolecules of interest during biological pretreatment by one or more fungal strains. Indeed, waste recovery is currently only implemented on an industrial scale if it is financially profitable, whether in terms of its energy return, since raw material has a negative purchase cost (i.e., the waste producer finances the person who processes it), or because it produces a financially valuable material.

To promote the recycling of textile waste, this invention provides for the simultaneous production of high-value biomolecules of interest, which could be an additional incentive for operators of biogas production units. These biomolecules can be used for industrial purposes (pigments, detergents, etc.) or for cosmetic, pharmaceutical, or therapeutic purposes, depending on biological pretreatment conditions.

The biomolecules that may be produced have been described above.

Alternatively, these biomolecules are preferably chosen from proteins and/or enzymes or biomolecules with a molecular weight of less than 5000 Da, preferably less than 1000 Da, such as flavourings, surfactants, or colourants and/or terpene derivatives.

Preferably, these biomolecules are recovered before the biologically pretreated fibrous substrate is transferred to the biogas production facility.

It is understood that this invention is in no way limited to the embodiments described above and that many modifications may be made thereto without departing from the scope of the appended claims

1. A method for producing biogas from a fibrous substrate comprising:
Biologically pretreating the fibrous substrate, forming a biologically pretreated fibrous substrate
Transferring the biologically pretreated fibrous substrate to a biogas production facility
Producing biogas from the pretreated fibrous substrate with the formation of a digestate,
Collecting the digestate
the method being characterised in that the biological pretreatment of the fibrous substrate is a solid-state fermentation using one or more filamentous fungal strains, more particularly using one or more saprophytic filamentous strains, of a woven, non-woven or agglomerated fibrous substrate, and shredded into pieces where said biologically pretreated fibrous substrate is a fibrous substrate shredded into pieces colonised by said one or more fungal strains.

2. The method for producing biogas from a fibrous substrate according to claim 1, wherein said one or more fungal strains is selected from the group consisting of strains belonging to the genera Agrocybe, Ganoderma, Trametes, Pycnoporus, Pleurotus, Fomes, Fomitopsis, Irpex, Laetiporus, Inonotus, Lentinula, Fusarium, Aspergillus, Trichoderma, Penicillium, Cladosporium, Chaetomium, and Acremonium.

3. The method for producing biogas from a fibrous substrate according to claim 1, wherein the agglomerated, woven, or non-woven shredded fibrous substrate comprises fibres chosen from natural plant or animal textile fibres, semi-synthetic textile fibres or polymer textile fibres, and lignocellulosic fibres.

4. The method for producing biogas from a fibrous substrate according to claim 1, wherein the agglomerated, woven, or non-woven shredded fibrous substrate is a residue from the crushing of recycled textiles, more particularly recycled furnishing textiles, recycled mattresses, bathroom or bed linen, clothing textiles, textile production scraps or waste, upholstery, and mixtures thereof.

5. The method for producing biogas from a fibrous substrate according to claim 1, wherein said fibrous substrate in agglomerated, woven or non-woven form is a residue from the crushing of recycled textiles chosen from furnishing, mattress and upholstery textiles and has a synthetic foam content of between 10 and 80%.

6. The method for producing biogas from a fibrous substrate according to claim 1, wherein said fibrous substrate in agglomerated, woven or non-woven form is a residue from the crushing of chipboard.

7. The method for producing biogas from a fibrous substrate according to claim 1, wherein said fibrous substrate comprises a proportion of synthetic fibres, semi-synthetic fibres, synthetic foam, such as for example PU foam, and natural plant or animal fibres, and wherein the collected digestate is enriched with synthetic fibres and plastic materials at a rate of more than 85% by weight relative to the weight of digestate.

8. The method for producing biogas from a fibrous substrate according to claim 1, wherein biologically pretreating the fibrous substract comprises:
conditioning said fibrous substrate to obtain a conditioned fibrous substrate containing from 60 to 80% by weight of water relative to the weight of conditioned fibrous substrate,
sanitising said conditioned fibrous substrate to form a sanitised conditioned fibrous substrate,
cooling the sanitised conditioned fibrous substrate for a period of time of between 12 and 24 hours,
inoculating said sanitised conditioned fibrous substrate with mycelium spawn from said one or more fungal strains on seeds by adding at least one mycelium spawn on seeds to said sanitised conditioned fibrous substrate at a rate of 0.5% to 10% by weight, more particularly 1 to 7%, more particularly 3 to 5% by weight of mycelium spawn relative to the weight of sterilised conditioned fibrous substrate, or inoculating from a liquid culture of said one or more fungal strains to obtain an inoculated sterilised conditioned fibrous substrate,
mixing said inoculated sanitised conditioned fibrous substrate to obtain a homogenised inoculated sanitised conditioned fibrous substrate,
incubating said homogenised inoculated sanitised conditioned fibrous substrate for a period of time of between 1 and 6 weeks, more particularly of between 2 and 5 weeks, in an enclosure having a relative humidity of between 65 and 85%, more particularly of between 70 and 80%, and
collecting a fibrous substrate colonised by said one or more fungal strains forming said biologically pretreated fibrous substrate.

9. The method for producing biogas from a fibrous substrate according to claim 8, wherein said sterilisation is steam pasteurisation so as to obtain a conditioned fibrous substrate sterilised for a period of at least 3 hours, preferably at least 4 hours, more preferably at least 5 hours at a temperature greater than or equal to 72° C.

10. The method for producing biogas from a fibrous substrate according to claim 8, wherein said sanitisation of the conditioned fibrous substrate is a composting comprising at least one composting cycle comprising a step of increasing the temperature until a temperature of between 55 and 80° C. is obtained, more preferably until a temperature of between 58 and 65° C. is obtained, for a period of time of between 6 hours and 5 days, followed by a step of ventilating said fibrous substrate so as to maintain a temperature of between 46 and 49° C. for 3 to 7 days, optionally by turning the fibrous substrate over.

11. The method for producing biogas from a fibrous substrate according to claim 8, wherein said step of conditioning said fibrous substrate to obtain a conditioned fibrous substrate comprises moistening the fibrous substrate and/or washing said fibrous substrate optionally followed by draining or drying and/or an additional step of supplementing with essential elements, such as for example minerals (calcium, magnesium), phosphorus, carbon and nitrogen sources, typically so as to obtain a fibrous substrate whose carbon: nitrogen ratio is between 10 and 30, preferably between 15 and 20, for example by adding grains.

12. The method for producing biogas from a fibrous substrate according to claim 1, comprising-simultaneously with pretreatment-a production of biomolecules, collected at the same time, said one or more fungal strains comprising at least one biomolecule-producing strain, wherein the said biomolecules are selected from the group consisting of proteins, enzymes, oligosaccharides, polysaccharides, antibiotics, antimitotics, antivirals, biosorbents and surfactants.

13. The method for producing biogas from a fibrous substrate according to claim 12, wherein said biomolecules are surfactants.

14. The method for producing biogas from a fibrous substrate according to claim 1, wherein the collected digestate is inactivated, more particularly inactivated using heat, and shaped, for example into blocks, modules, or panels.

15. The method for producing biogas from a fibrous substrate according to claim 1, further comprising the step of recycling synthetic fibres and/or plastics present in the collected digestate.

16. Method to produce one or more biomolecules from a fibrous substrate comprising:
Biologically pre-treating the fibrous substrate, forming a biologically pretreated fibrous substrate,
Producing the biomolecule from the pretreated fibrous substrate with the formation of a digestate,
Collecting the digestate and
Collecting the said biomolecule(s), wherein the said biomolecule(s) is selected from the group consisting of oligosaccharides, polysaccharides, antibiotics, antimitotics, antivirals, biosorbents, surfactants, proteins and/or enzymes and mixture thereof,
wherein the biological pretreatment is a solid-state fermentation using one or more filamentous fungal strains, more particularly using one or more saprophytic filamentous strains,
wherein the fibrous substrate is a woven, non-woven or agglomerated fibrous substrate and shredded into pieces,
wherein the non-woven shredded fibrous substrate comprises fibres chosen from natural plant or animal textile fibres, semi-synthetic textile fibres or polymer textile fibres, and lignocellulosic fibres, or
wherein the agglomerated, woven, or non-woven shredded fibrous substrate is a residue from the crushing of recycled textiles, more particularly recycled furnishing textiles, recycled mattresses, bathroom or bed linen, clothing textiles, textile production scraps or waste, upholstery, and mixtures thereof, or
wherein said fibrous substrate in agglomerated, woven or non-woven form is a residue from the crushing of recycled textiles chosen from furnishing, mattress and upholstery textiles and has a synthetic foam content of between 10 and 80%, or
wherein said fibrous substrate in agglomerated, woven or non-woven form is a residue from the crushing of lignocellulosic elements, such as for example residue from the crushing of chipboard, or
wherein said fibrous substrate comprises a proportion of synthetic fibres, semi-synthetic fibres, synthetic foam, such as for example PU foam, and natural plant or animal fibres, and wherein the collected digestate is enriched with synthetic fibres and plastic materials at a rate of more than 85% by weight relative to the weight of digestate.

17. The method to produce the one or more biomolecules from a fibrous substrate of claim 16, wherein the biomolecules have a molecular weight below 5000 Da, preferably below 1000 Da.

18. The method to produce the one or more biomolecules from a fibrous substrate of claim 16 further comprising the step of recycling synthetic fibres and/or plastics present in the collected digestate.

19. The method to produce one or more biomolecules from a fibrous substrate of claim 16, wherein the biomolecule is a surfactant.

20. The method to produce one or more biomolecules from a fibrous substrate of claim 19, wherein the surfactant is recovered upon acid precipitation at a pH at or below 4.0 (preferably below 3, or even below 2.5), followed by the step of resuspending the filtrate in a medium at a pH at or above 7.0 and to separate the resuspended surfactant from impurities using a filtration with a 100 kDa cutoff, followed by a nanofiltration, using a 1 kDa cutoff membrane.

21. The method to produce one or more biomolecules from a fibrous substrate of claim 19, wherein the surfactants are selected from the group consisting of sophrolipids, mannosylerythritol lipids (MEL), trehalose lipids, xylolipids, cellobioses lipids, polylipids, lipopeptides, hydrophobins and a mixture thereof.
